(19) United States
(12) Patent Application Publication
(10) Pub. No.: US 2012/0122913 A1
(43) Pub. Date: May 17, 2012

(54) COMBINATION OF CURCUMINOIDS AND MTOR INHIBITORS FOR THE TREATMENT OF TAUOPATHIES

(75) Inventors: Michel Charbonneau, Laval (CA); Guylaine Lassonde, Laval (CA)

(73) Assignee: Institut National De La Recherche Scientifque

(21) Appl. No.: 13/386,665

(22) PCT Filed: Jul. 14, 2010
(86) PCT No.: PCT/CA2010/001086

§ 371 Date: Jan. 23, 2012

Related U.S. Application Data
(60) Provisional application No. 61/228,257, filed on Jul. 24, 2009.

(51) Int. Cl. A61K 31/436; A61P 25/28; C12N 5/079; A61K 31/12

(52) U.S. Cl. 514/291; 514/679; 435/375

(57) ABSTRACT

Methods, uses, compositions, combinations and kits relating to the decrease of Tau protein levels, and prevention and/or treatment of diseases or disorders associated with Tau protein (Tauophathies), such as Alzheimer's disease, using a curcuminoid and a mammalian target rapamycin (mTOR) inhibitor, are described.

### CROSS REFERENCE TO RELATED APPLICATIONS

This application claims the benefit of U.S. provisional application Ser. No. 61/228,257, filed Jul. 24, 2009, the content of which is incorporated herein by reference in its entirety.

### TECHNICAL FIELD

The present invention relates to the prevention and/or treatment of tauopathies using a combination therapy.

### BACKGROUND ART

Many neurological diseases such as Alzheimer's disease (AD) and some forms of dementias have the common pathological feature of neurofibrillary tangles (NFTs), which are structures made up of paired helical filaments (PHFs), composed primarily of tau protein in a hyperphosphorylated state. Such diseases are typically referred to as tauopathies. Tau belongs to a family of microtubule associated proteins (MAPs) that are thought to be involved in stabilizing and remodeling the neuronal cytoskeleton. Under normal physiological conditions, tau is found primarily in the brain, associated with the cellular cytoskeleton. Abnormal tau phosphorylation and deposition in neurons and glial cells is one of the major features in tauopathies such as Alzheimer's Disease (AD). The adult human brain expresses six different isoforms of tau protein, ranging in size from 352 to 441 amino acid residues (Entrez GeneID 4137 for human). The isoforms differ by having either three or four microtubule binding domains, and by the presence or absence of amino-terminal inserts. Tau is phosphorylated in the brain by a variety of kinases.

Alzheimer's Disease is characterized by a progressive decline in cognitive function. Neuropathologies of the disease include the accumulation of tangles, β-amyloid containing plaques, dystrophic neurites, and loss of synapses and neurons (Selkoe, D. et al., 1999, Alzheimer's Disease, 2^{nd} Ed., Terry R. et al., eds. pg. 293-310. Philadelphia: Lippincott, Williams and Wilkins).

Only a limited number of pharmacological agents have been identified as effective in treating symptoms of AD. The most prominent of these today are tacrine and donepezil hydrochloride, which are cholinesterase inhibitors active in the brain. These drugs do not slow the progress of the disease. Furthermore no compound has been established as effective in blocking the development or progression of AD.

There is thus a need for novel compositions and methods for the treatment of tauopathies such as AD.

The present description refers to a number of documents, the content of which is herein incorporated by reference in their entirety.

### SUMMARY OF THE INVENTION

The present invention relates to the prevention and/or treatment of tauopathies using a combination therapy.

In an aspect, the present invention provides a method of preventing or treating a tauopathy in a subject, said method comprising administering a curcuminoid and a mammalian target of rapamycin (mTOR) inhibitor to said subject.

In another aspect, the present invention provides a method of preventing or treating a tauopathy, said method comprising administering in a subject in need thereof an effective amount of a curcuminoid and an effective amount of a mammalian target of rapamycin (mTOR) inhibitor.

In an embodiment, the above-mentioned curcuminoid and mTOR inhibitor are administered simultaneously.

In an embodiment, the above-mentioned curcuminoid and mTOR inhibitor are administered sequentially.

In an embodiment, the above-mentioned method comprises administering a composition comprising said curcuminoid and said mTOR inhibitor.

In an embodiment, the above-mentioned method comprises administering a first composition comprising the curcuminoid and a pharmaceutically acceptable carrier, and a second composition comprising the mTOR inhibitor and a pharmaceutically acceptable carrier.

In a further aspect, the present invention provides a method of decreasing the level of Tau protein in a cell, tissue or organ, said method comprising contacting said cell, tissue or organ with a curcuminoid and an mTOR inhibitor.

In a further aspect, the present invention provides a kit comprising a curcuminoid, an mTOR inhibitor, and instructions for treating a tauopathy in a subject, or for decreasing the level of Tau protein in a cell, tissue or organ.

In a further aspect, the present invention provides a composition for use in treating a tauopathy in a subject, or for use in decreasing the level of Tau protein in a cell, tissue or organ, said composition comprising a curcuminoid and an mTOR inhibitor. In an embodiment, the above-mentioned composition further comprises a pharmaceutically acceptable carrier, excipient and/or diluent.

In a further aspect, the present invention provides a use of a curcuminoid and an mTOR inhibitor for preventing or treating a tauopathy in a subject.

In a further aspect, the present invention provides a use of a curcuminoid and an mTOR inhibitor for the preparation of a medicament.

In a further aspect, the present invention provides a use of a curcuminoid and an mTOR inhibitor for the preparation of a medicament for preventing or treating a tauopathy in a subject.

In a further aspect, the present invention provides a use of a first composition comprising a curcuminoid and a pharmaceutically acceptable carrier, excipient and/or diluent, and a second composition comprising an mTOR inhibitor and a pharmaceutically acceptable carrier, excipient and/or diluent, for preventing or treating a tauopathy in a subject.

In a further aspect, the present invention provides a use of a curcuminoid and an mTOR inhibitor for decreasing the level of Tau protein in a cell, tissue or organ.

In a further aspect, the present invention provides a use of a curcuminoid and an mTOR inhibitor for the preparation of a medicament for decreasing the level of Tau protein in a cell, tissue or organ.

In a further aspect, the present invention provides a use of a first composition comprising a curcuminoid and a pharmaceutically acceptable carrier, and a second composition comprising an mTOR inhibitor and a pharmaceutically acceptable carrier, for decreasing the level of Tau protein in a cell, tissue or organ.

In a further aspect, the present invention provides a combination for use in preventing or treating a tauopathy in a subject, or for use in decreasing the level of Tau protein in a cell, tissue or organ, said combination comprising a curcuminoid and an mTOR inhibitor.

In a further aspect, the present invention provides a combination for use in preventing or treating a tauopathy in a subject, or for use in decreasing the level of Tau protein in a cell, tissue or organ, said combination comprising a first composition comprising a curcuminoid and a pharmaceutically acceptable carrier, and a second composition comprising an mTOR inhibitor and a pharmaceutically acceptable carrier.

In an embodiment, the above-mentioned curcuminoid is curcumin or a derivative thereof.

In an embodiment, the above-mentioned curcuminoid is curcumin.

In an embodiment, the above-mentioned mTOR inhibitor is a rapamycin derivative.

In an embodiment, the above-mentioned rapamycin derivative is Everolimus or Temsirolimus.

In an embodiment, the above-mentioned tauopathy is Alzheimer's disease.

In an embodiment, the above-mentioned subject is a mammal, in a further embodiment, a human.

In an embodiment, the above-mentioned cell, tissue or organ is a neuronal cell, tissue or organ.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the effect of a 48 h treatment with curcumin (20 μM) and Everolimus (Evero, 2 nM), alone or in combination, on total Tau protein recovered in differentiated SH-SY5Y cells concomitantly exposed to 5 nM of β-amyloid₍₁₋₄₂₎. Data are mean±SEM;

FIG. 2 shows the effect of a 48 h treatment with curcumin (20 μM) and Everolimus (Evero, 2 nM), alone or in combination, on the viability of differentiated SH-SY5Y cells concomitantly exposed to 5 nM of β-amyloid₍₁₋₄₂₎. Data are mean±SEM;

FIG. 3 shows the effect of a 48 h treatment with curcumin (10 μM) and Everolimus (Evero, 1 nM), alone or in combination, on total Tau protein recovered in differentiated SH-SY5Y cells concomitantly exposed to 5 μM of β-amyloid₍₁₋₄₂₎. Data are mean±SEM;

FIG. 4 shows the effect of a 48 h treatment with curcumin (10 μM) and Everolimus (Evero, 1 nM), alone or in combination, on the viability of differentiated SH-SY5Y cells concomitantly exposed to 5 μM of β-amyloid₍₁₋₄₂₎. Data are mean±SEM;

FIG. 5 shows the effect of a 48 h treatment with curcumin (15 μM) and Temsirolimus (Temsiro, 2 nM), alone or in combination, on total Tau protein recovered in differentiated SH-SY5Y cells concomitantly exposed to 5 nM of β-amyloid₍₁₋₄₂₎. Data are mean±SEM; and

FIG. 6 shows the effect of a 48 h treatment with curcumin (15 μM) and Temsirolimus (Temsiro, 2 nM), alone or in combination, on the viability of differentiated SH-SY5Y cells concomitantly exposed to 5 nM of β-amyloid₍₁₋₄₂₎. Data are mean±SEM.

### DISCLOSURE OF INVENTION

The present inventors have determined that the combination of a curcuminoid (curcumin) with a mammalian target of rapamycin (mTOR) inhibitor (Everolimus or Temsirolimus) is effective at reducing the levels of Tau protein in human neuron-like cells.

Accordingly, in an aspect, the present invention provides a method of treating a tauopathy in a subject, said method comprising administering a curcuminoid and a mammalian target of rapamycin (mTOR) inhibitor to said subject.

In another aspect, the present invention provides a method of treating a tauopathy in a subject, said method comprising administering a first composition comprising a curcuminoid and a pharmaceutically acceptable carrier, and a second composition comprising an mTOR inhibitor and a pharmaceutically acceptable carrier, to said subject.

In another aspect, the present invention provides a combination for treating a tauopathy in a subject, said combination comprising a curcuminoid and a mammalian target of rapamycin (mTOR) inhibitor.

In another aspect, the present invention provides a method of decreasing the level of Tau protein (of one ore more isoforms of Tau) in a cell, said method comprising contacting said cell with a curcuminoid and an mTOR inhibitor.

In another aspect, the present invention provides a combination for decreasing the level of Tau protein in a cell, said combination comprising a curcuminoid and an mTOR inhibitor.

In another aspect, the present invention provides a kit comprising a curcuminoid and an mTOR inhibitor together with instructions for treating a tauopathy in a subject, or for decreasing the level of Tau protein in a cell.

In another aspect, the present invention provides a composition for treating a tauopathy in a subject, or for decreasing the level of Tau protein in a cell, said composition comprising a curcuminoid and an mTOR inhibitor.

In another aspect, the present invention provides a combination for treating a tauopathy in a subject, or for decreasing the level of Tau protein in a cell, tissue or organ, said combination comprising a curcuminoid and an mTOR inhibitor.

In another aspect, the present invention provides a combination for treating a tauopathy in a subject, or for decreasing the level of Tau protein in a cell, tissue or organ, said combination comprising a first composition comprising a curcuminoid and a pharmaceutically acceptable carrier, and a second composition comprising an mTOR inhibitor and a pharmaceutically acceptable carrier.

In another aspect, the present invention provides a use of a curcuminoid and an mTOR inhibitor, or of the above-noted composition, for treating a tauopathy in a subject.

In another aspect, the present invention provides a use of a curcuminoid and an mTOR inhibitor for the preparation of a medicament for treating a tauopathy in a subject.

In another aspect, the present invention provides a use of a curcuminoid and an mTOR inhibitor, or of the above-noted composition, for decreasing the level of Tau protein in a cell.

In another aspect, the present invention provides a use of a curcuminoid and an mTOR inhibitor for the preparation of a medicament for decreasing the level of Tau protein in a cell.

In another aspect, the present invention provides a use of a curcuminoid and an mTOR inhibitor, or of the above-noted composition, for treating a tauopathy in a subject.

In another aspect, the present invention provides a use of the above-noted combination for treating a tauopathy in a subject, for decreasing the level of Tau protein in a cell, for the preparation of a medicament for treating a tauopathy in a subject, or for the preparation of a medicament for decreasing the level of Tau protein in a cell.

In an embodiment, the above-mentioned Tau protein is a phosphorylated (e.g., hyperphosphorylated) form of one ore more isoforms of Tau.

“Curcuminoid” as used herein refers to any compound have the general structure the curcuminoid genus, and which possesses an activity (e.g., a biological activity) similar to that of curcumin. In an embodiment, the curcuminoid has the structure of formula (I)

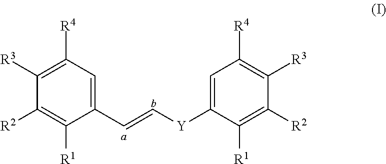

wherein
R¹ is independently H, OAc, OCH₃, or F;
R² is independently H or OCH₃;
R³ is independently H, OH, NO₂, OCH₃, or COOH;
R⁴ is independently H or OCH₃;
the double bond between the carbons labelled a and b may either be a trans double bond or a cis double bond; and
Y is

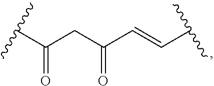

or its enol form

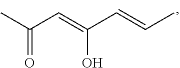

or the cyclic forms

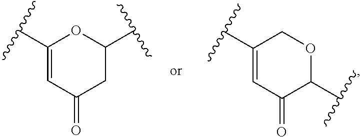

or the monoketo forms

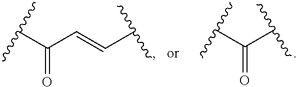

The wavy lines in the Y structures above represent the points of attachment to carbon b (to the left of Y in formula I above) and the phenyl ring structure (to the right of Y in formula I above) of formula I. For example, when Y is the first structure noted above, i.e.,

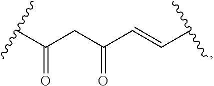

the resulting curcuminoid is as follows (formula II):

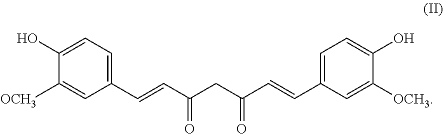

Similarly, the third and fourth Y structures noted above (cyclic forms) would result in curcuminoids of formulae VI and VII, respectively, indicated below.

In an embodiment, the above-mentioned curcuminoid is a curcumin or a derivative thereof which has been modified to alter one of its physical-chemical properties, for example to increase its solubility and/or bioavailability.

Representative curcuminoids include, for example, curcumin (formula II, as well as its enol form as per formula IIa), demethoxycurcumin (formula III, as well as its enol form as per formula IIIa), bisdemethoxycurcumin (formula IV, as well as its enol form as per formula IVa), cis-trans curcumin (formula V, as well as its enol form as per formula Va) as well as cyclocurcumin (both formulae VI and VII have been described).

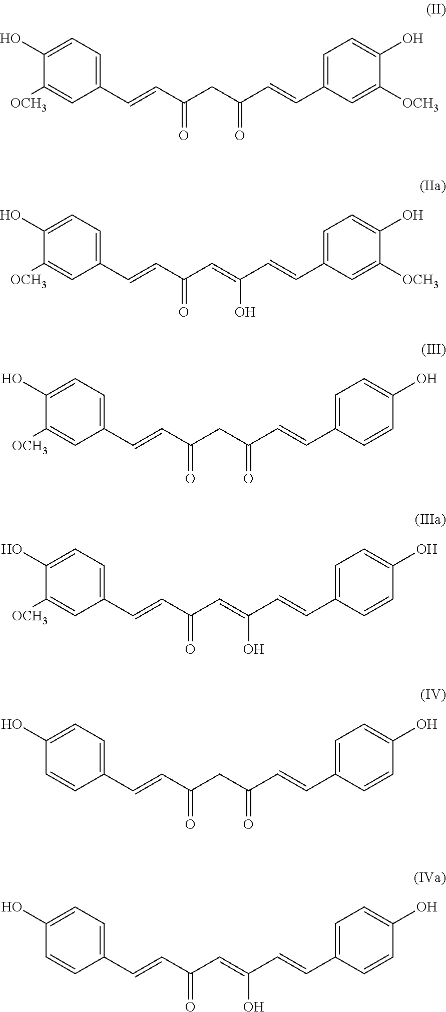

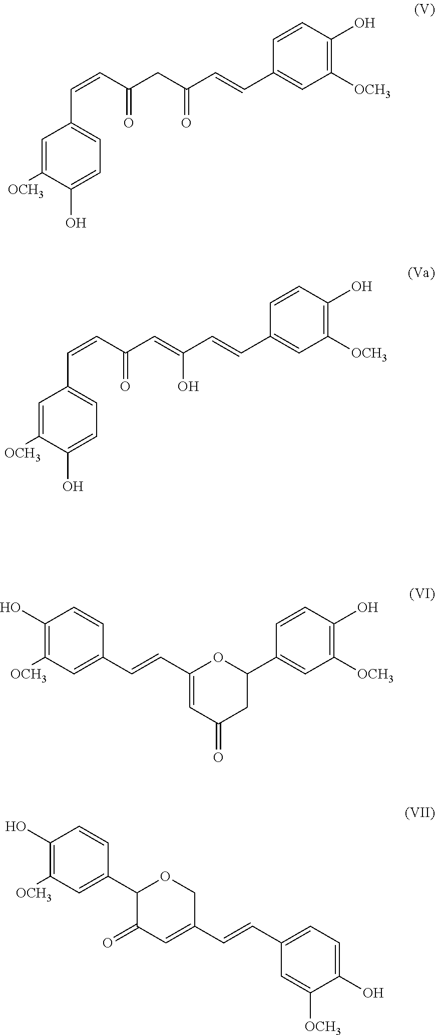

The diketo forms portion of the backbone described above may give rise to tautomeric isomers, i.e., structures VIII or VIIIa:

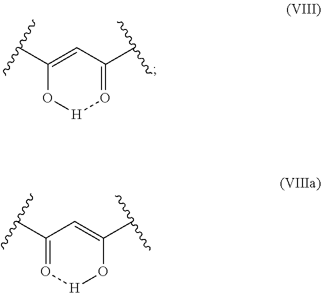

Further, forms of curcuminoids having a saturated alkyl chain, i.e., in which a double bond is replaced by a single bond in one or both of the chains connecting the central di-keto or enol structure above to the terminal phenyl ring portions (e.g., replacement of the double bond between carbons a and b as noted above or the double bond in the first two Y structures noted above, by a single bond). An example of such a form has been referred to as tetrahydrocurcumin (see for example Dinkova-Kostova, A. T. et al. (1999), Carcinogenesis 20(5): 911-914).

Forms in which the diketo/enol structure is replaced with for example a piperidone-based structure, a hydrazino-based structure or a bis-alkynyl heteroaromatic-type structure have also been described (see Mosley et al. (2007) Adv. Exp. Med. Biol. 595: 77-103).

A number of curcuminoids are described for example in Aggarwal, B. B. et al., Chapter 10: Curcumin—Biological and Medicinal Properties, in Turmeric: The genus Curcuma, Ravindran, P. N. et al. (eds.), pp. 297-368, CRC Press, Boca Raton, Fla., 2007; Dinkova-Kostova, A. T. et al. (supra); and Mosley et al. (supra).

The term curcuminoid as used herein includes any derivatives, analogs, prodrugs and metabolites of curcuminoids (such as those described in the references noted above as well as in PCT publication Nos. WO 09/023357, WO 09/017874, WO 08/066151, WO 08/051474, WO 08/045534, WO 07/098504, WO 07/051314, WO 06/044379, WO 04/031122, WO 03/105751, WO 03/088927, WO 02/002582 and WO 01/040188), and curcuminoid glycosides (such as those described in PCT publication No. WO 05/007667). It also includes any salt (e.g., pharmaceutically acceptable salts) of the above-mentioned curcuminoids. Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids when the curcuminoid contains a suitable basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when the curcuminoid contains a suitable acidic moiety.

Curcuminoid useful in the method, uses, kits and compositions of the present invention may be obtained and isolated from many sources, or chemically synthesized using well known methods. For example, curcumin (also referred to as Diferuloylmethane, Diferulylmethane, Natural Yellow 3 and (E,E)-1,7-bis(4-Hydroxy-3-methoxyphenyl)-1,6-heptadiene-3,5-dione) is a yellow pigment found in the rhizome of Curcuma longa L., the source of the spice turmeric, and may thus be isolated from this source using methods well known in the art (see, for example, PCT publication No. WO 02/16503). Curcuminoids may also be obtained from other botanicals in addition to Curcuma longa L., such as Curcuma xanthorrhiza Roxb. and Curcuma zedoaria Rosc. Curcumin may also be obtained from commercial sources such as from Sigma-Aldrich (Cat No. C7727). The curcumin analogs demethoxycurcumin, bisdemethoxycurcumin and tetrahydrocurcumin can also be obtained from many sources, or readily prepared from curcumin by those skilled in the art. Methods to prepare curcuminoids are described, for example, in PCT publication Nos. WO 06/089894 and WO 08/123390, U.S. Pat. Nos. 5,679,864, 6,790,979 and 7,507,864; Pedersen et al. (1985) Chem. Abstract. 103:178092; and Nurfina et al. (1997) Eur. J. Med. Chem. 32: 321-328.

The term “mTOR inhibitor” as used herein refers to any compound capable of inhibiting the expression and/or activity of the mammalian target of rapamycin (mTOR) protein (also known as FK506 binding protein 12-rapamycin associated protein 1 (FRAP1)), and more particularly of the mTOR Complex 1 (mTORC1). MTORC1 comprises at least four proteins, namely mTOR, regulatory associated protein of mTOR (Raptor), mammalian LST8/G-protein β-subunit like protein (mLST8/GβL) and proline-rich Akt substrate of 40 kDa (PRAS40).

A representative mTOR inhibitor is the macrolide rapamycin (also known as sirolimus, Rapamune™, which is a product of Streptomyces hygroscopicus. It has the following structure:

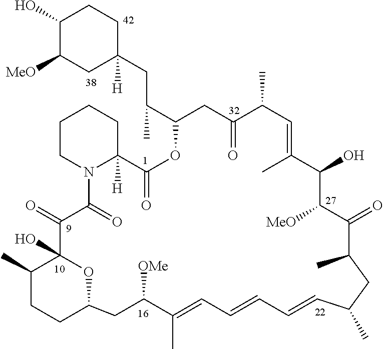

mTOR inhibitors also include any analog, derivative, prodrug or metabolite of rapamycin, such as esters, ethers, oximes, hydrazones, and hydroxylamines of rapamycin, as well as rapamycins in which functional groups on the rapamycin nucleus have been modified, for example through reduction or oxidation.

Esters and ethers of rapamycin include, for example, alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. Nos. 5,411,967, 5,434,260, 5,480,988, 5,480,989 and 5,489,680); hindered N-oxide esters (U.S. Pat. No. 5,491,231); biotin esters (U.S. Pat. No. 5,504,091); 0-alkyl ethers (U.S. Pat. No. 5,665,772); and PEG esters of rapamycin (U.S. Pat. No. 5,780,462). The preparation of these esters and ethers are disclosed in the patents listed above.

Oximes, hydrazones, and hydroxylamines of rapamycin are disclosed, for example, in U.S. Pat. Nos. 5,373,014, 5,378,836, 5,023,264, and 5,563,145. The preparation of these oximes, hydrazones, and hydroxylamines are disclosed in the above listed patents.

The term “mTOR inhibitor” also includes any salt (e.g., pharmaceutically acceptable salts) of the above-mentioned mTOR inhibitor (e.g., rapamycin as well as derivatives, analogs, prodrugs or metabolites thereof). Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable aids when the mTOR inhibitor contains a suitable basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when the mTOR inhibitor contains a suitable acidic moiety.

In an embodiment, the above-mentioned rapamycin derivative is Everolimus (also known as RAD-001, Certican™, and Afinitor™) or Temsirolimus (also known as CCI-779 and Torisel™). In a further embodiment, a combination of mTOR inhibitors may be used, such as a combination of rapamycin derivatives, for example a combination of Everolimus and Temsirolimus.

The invention further provides a composition comprising a curcuminoid and a pharmaceutically acceptable diluent or carrier; a composition comprising an mTOR inhibitor and a pharmaceutically acceptable diluent or carrier; a composition comprising a curcuminoid and an mTOR inhibitor; and a composition comprising a curcuminoid, an mTOR inhibitor and a pharmaceutically acceptable diluent or carrier.

In addition to the active ingredients (e.g., a curcuminoid, an mTOR inhibitor, or both), pharmaceutical compositions may contain suitable pharmaceutically-acceptable carriers comprising excipients and auxiliaries, which facilitate processing of the active compounds into preparations, which can be used pharmaceutically. When the excipient serves as a diluent, it can be a solid, semisolid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions, and sterile packaged powders (see Remington: The Science and Practice of Pharmacy by Alfonso R. Gennaro, 2003, 21^{th} edition, Mack Publishing Company).

Some examples of suitable excipients include lactose, dextrose, sucrose, sorbitol, mannitol, starches, lecithin, phosphatidylcholine, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate, and mineral oil; wetting agents; emulsifying and suspending agents; preserving agents such as methyl- and propylhydroxybenzoates; sweetening agents; and flavoring agents. The compositions of the invention can be formulated so as to provide quick sustained or delayed release of the active ingredient after administration to the patient by employing procedures known in the art.

Pharmaceutical compositions/formulations comprising a curcuminoid are well known in the art. Curcuminoid formulations having enhanced bioavailability are described, for example, in PCT publication No. WO 07/103435. Bioavailability can also be enhanced using a phospholipid complex (using phosphatidyl choline, phosphatidyl serine or phosphatidyl ethanolamine, for example) as described for example in published European application no. EP 1 837 030. Curcuminoid may be formulated/encapsulated with colloidal drug delivery vehicles (e.g., lipid-based and polymer-based particles, such as nanoparticles, nanocapsules/nanospheres, microparticles/microspheres, block copolymer micelles and liposomes) using methods well known in the art (see, for example, Bisht, Savita; et al. (2007) Journal of Nanobiotechnology 5 (3): 3; Begum et al. (2008) J Pharmacol Exp Ther 326(1): 196). Encapsulated curcuminoid formulations, such as liposomal curcuminoid formulations, are described, for example, in U.S. Patent publication No. 2008/0138400.

Pharmaceutical compositions/formulations comprising an mTOR inhibitor (e.g., rapamycin) are well known in the art. A variety of oral and parenteral dosage forms are known for rapamycin and a number of rapamycin analogs. Some are currently in use in various treatment methods, monotherapies or otherwise. Those same dosage forms may likewise be used in the practice of the mTOR inhibitor-based therapy disclosed herein. Solid dosage forms are often preferred for oral administration and include among others conventional admixtures, solid dispersions and nanoparticles, typically in tablet, capsule, caplet, gel cap or other solid or partially solid form. Such formulations may optionally contain an enteric coating. Numerous materials and methods for such oral formulations are well known. A typical example of the use of conventional materials and methods to formulate an mTOR inhibitor is shown in U.S. Patent publication No. US 2004/0077677 and PCT publication No. WO 04/026280, as well as in U.S. Pat. Nos. 6,197,781; 6,589,536; 6,555,132; 5,985,321; 6,565,859; 5,932,243; 5,559,121; 5,536,729; 5,989,591 and 5,985,325.

Pharmaceutical compositions suitable for use in the invention include compositions wherein the active ingredients are contained in an effective amount to achieve the intended purpose (e.g., preventing and/or ameliorating and/or inhibiting a disease). The determination of an effective dose is well within the capability of those skilled in the art. For any compounds, the therapeutically effective dose can be estimated initially either in cell culture assays (e.g., cell lines) or in animal models, usually mice, rabbits, dogs or pigs. The animal model may also be used to determine the appropriate concentration range and route of administration. Such information can then be used to determine useful doses and routes for administration in humans. An effective dose or amount refers to that amount of one or more active ingredient(s), for example a curcuminoid and an mTOR inhibitor, which is sufficient for treating a specific disease or condition (e.g., a tauopathy). Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Guidance as to particular dosages and methods of delivery is provided in the literature and generally available to practitioners in the art.

The term “tauopathy” as used herein refers to a disease, condition or disorder associated with the expression/overexpression, accumulation and/or aggregation of a Tau protein (e.g., of a given form/isoform of a Tau protein, such as a hyperphosphorylated form) in a given cell, tissue or organ of a subject, and includes diseases/conditions such as Alzheimer's disease, argyrophilic grain disease, frontotemporal dementia, progressive supranuclear palsy, corticobasal degeneration as well as frontotemporal lobar degeneration (also known as Pick's disease). In an embodiment, the above-mentioned expression/overexpression, accumulation and/or aggregation is in a central nervous system (CNS) cell, tissue or organ, such as the brain (e.g., neurons, glial cells). In an embodiment, the above-mentioned tauopathy is a neurodegenerative disease, condition or disorder. In a further embodiment, the above-mentioned tauopathy is Alzheimer's disease.

“Treatment” or “treating” a disease (e.g., a tauopathy) as used herein refers to the administration of one or more compound(s) to elicit a desired therapeutic medicinal/biological response in a tissue, system, animal, individual or human, in order to have one or more of the following effects:

(A) Inhibiting the disease; for example, inhibiting a disease, condition or disorder associated with a Tau protein (e.g., expression, accumulation and/or aggregation of a Tau protein) in an individual that is experiencing or displaying the pathology or symptomatology of the disease, condition or disorder (i.e., arresting further development of the pathology and/or symptomatology); and

(B) Ameliorating the disease; for example, ameliorating disease, condition or disorder associated with a Tau protein (e.g., expression, accumulation and/or aggregation of a Tau protein) in an individual that is experiencing or displaying the pathology or symptomatology of the disease, condition or disorder (i.e., reversing the pathology and/or symptomatology).

The expression “decreasing the level of Tau protein in a cell, tissue or organ” as used herein refers to a decrease in the level of one or more isoforms of a Tau protein in the cell, tissue or organ. It also refers to a decrease on the level of aggregated Tau protein in the cell, tissue or organ. In an embodiment, the above-mentioned isoform of a Tau protein is a phosphorylated or hyperphosphorylated isoform.

The present invention relates to the administration or co-administration of a curcuminoid and an mTOR inhibitor (or a composition comprising one or both of these agents), to elicit any of the effects discussed above. The curcuminoid and an mTOR inhibitor may be administered separately or in combination (e.g., together in a composition). The combination of therapeutic agents and compositions of the present invention may be administered or co-administered in any conventional dosage form, as discussed above. Co-administration in the context of the present invention refers to the administration of more than one therapeutic in the course of a coordinated treatment to achieve an improved clinical outcome. Such co-administration may also be coextensive, that is, occurring during overlapping periods of time. For example, the curcuminoid may be administered to a patient before, concomitantly, before and after, or after the mTOR inhibitor is administered (or vice versa).

In an embodiment, the above-mentioned method, use or composition further comprises one or more additional active agent(s) (e.g., an agent currently used for the treatment of a tauopathy).

As used herein, a synergistic effect (e.g., reduction in the amount and/or aggregation of a Tau protein) is achieved when the effect of the combined drugs is greater than the theoretical sum of the effect of each agent alone. One potential advantage of combination therapy with a synergistic effect is that lower dosages of one or both of the drugs or therapies may be used in order to achieve high therapeutic activity with low toxicity (e.g., a lower dose of a curcuminoid and/or an mTOR inhibitor provides therapeutic tauopathy activity with lower toxicity). Another potential advantage of combination therapy with a synergistic effect is that a more potent/efficacious therapeutic effect may be achieved. In an embodiment, the combination therapy results in at least a 5% increase in the effect as compared to the predicted theoretical additive effect of the agents. In a further embodiment, the combination therapy results in at least a 10% increase in the effect as compared to the predicted theoretical additive effect of the agents. In a further embodiment, the combination therapy results in at least a 20% increase in the effect as compared to the predicted theoretical additive effect of the agents. In a further embodiment, the combination therapy results in at least a 30% increase in the effect as compared to the predicted theoretical additive effect of the agents. In a further embodiment, the combination therapy results in at least a 50% increase in the effect as compared to the predicted theoretical additive effect of the agents.

The present invention further provides a kit or package comprising an agent, combination of agents (e.g., a curcuminoid and/or an mTOR inhibitor) or composition(s) of the present invention. The arrangement and construction of such kits is conventionally known to one of skill in the art. Such kits may include, for example, container(s) (e.g., a syringe and/or vial and/or ampoule) for containing the agent or combination of agents or compositions, other apparatus for administering the therapeutic agent(s) and/or composition(s) and/or diluent(s). The kit may optionally further include instructions. The instructions may describe how the agent(s) and the diluent should be mixed to form a pharmaceutical formulation. The instructions may also describe how to administer the resulting pharmaceutical formulation to a subject.

In an embodiment, the above-mentioned kit comprises instructions for the treatment of a tauopathy (e.g., Alzheimer's disease) in a subject, or for decreasing the level of Tau protein in a cell.

As used herein, the terms “subject” or “patient” are used interchangeably are used to mean any animal, such as a mammal, including humans and non-human primates. In an embodiment, the above-mentioned subject is a mammal. In a further embodiment, the above-mentioned subject is a human.

### MODE(S) FOR CARRYING OUT THE INVENTION

The present invention is illustrated in further detail by the following non-limiting examples.

### EXAMPLE 1

### Materials and Methods

The biological model used is the differentiated (6 days with retinoic acid and 2 days with Brain Derived Neurotrophic Factor (BDNF)) (Encinas et al., J Neurochem 2000, 75(3): 991-1003). On day 0, Petri dishes (5 cm in diameter) were seeded with 900,000 cells and 4 ml of culture medium (45% MEME, Sigma Cat. No. M5650) supplemented with 2 mM L-glutamine, 1 mM sodium pyruvate, 45% of nutrient mixture F12 Ham Kaighn's modification (F12K) (Sigma Cat. No. N3520) and 10% Fetal Bovine Serum (FBS) were added. For the cytotoxicity assay, 48 well-plates were seeded with 180,000 cells and 500 μl of culture medium was added. Five Petri dishes (or 6-well plates) were prepared for each treatment. On day 1 to 6 (inclusively), the culture medium was completely removed from Petri dishes and replaced with 3 ml (or 250 μL for wells) of fresh media 49.5% MEME (Sigma Cat. No. M5620) supplemented with 2 mM L-glutamine, 1 mM sodium pyruvate, 49.5% F12K (Sigma Cat. No. N3520) and 1% Fetal Bovine Serum) containing 10 μM of retinoic acid (Sigma Cat. No. R2625, from a 10 mM master solution in 100% ethanol). On day 7, the culture medium was removed and changed with a medium free of retinoic acid and FBS, but containing 2 nM of BDNF (Sigma Cat. No. B3795), from a 2 μM master solution in filtered sterile water). Chemical treatments were initiated and kept for 48 h. In a typical protocol, 8 groups were prepared, i.e. controls, curcumin only (Sigma cat. no. C7727, e.g., 20 nM from a master solution of 20 μM in 100% dimethyl sulfoxide), Everolimus (Sigma cat. no. 07741) or Temsirolimus (LC Laboratories, cat. no. T-8040) only (e.g., 2 nM from a master solution of 2 μM in 100% dimethyl sulfoxide) and a combination of curcumin and Everolimus or Temsirolimus, in the presence or absence of either 5 nM or 5 μM β-amyloid. The latter was used to mimic the cellular content of brain cells from patients with Alzheimer's Disease where β-amyloid plaques are present. The Human Beta Amyloid (Aβ₍₁₋₄₂₎, Chemicon Cat. No. AG972) solution was prepared according to the manufacturer's instructions. In brief, a master solution of 1 mg/ml (221.5 μM) of Aβ₍₁₋₄₂₎ in filtered sterile 1% NH₄OH was sonicated for 30 seconds, then diluted with sterile 10× PBS plus sterile water and 1 N HCl to obtain a 55 μM Aβ₍₁₋₄₂₎ solution at pH 7.4. The solution was subsequently diluted in serum-deprived culture medium containing BDNF (2 nM final concentration) to obtain either a 5 nM or 5 μM Aβ₍₁₋₄₂₎ final concentration. The solution for the control group was prepared in a similar manner without Aβ₍₁₋₄₂₎.

On day 8, cells were observed under an inverted microscope to ensure that they are fully adherent and free of microbial contamination. On day 9, Petri dishes were used for Western Blot analyses. The medium was removed, 4 ml of ice-cold PBS was added then removed from Petri dishes, and 75 μl of cell lysing buffer (modified from Jämsä et al. (2004) Biochem Biophys Res Commun 319(3): 993-1000): 50 mM Tris-HCl, pH 8, 150 mM NaCl, 1% Triton, 1 mM EDTA, 25 mM NaF, 0.01 mg/ml Aprotinin, 0.2 mg/ml PMSF, 1 mM Na₃VO₄) was added. Cells were then removed with a cell scraper, transferred to a 0.6 ml microtube and kept on ice. Samples were sonicated for 30 seconds and mixed with a vortex for 5 seconds and the procedure was repeated once. Samples obtained were stored at −80 ° C. in aliquots of 20 μl. For SDS-PAGE analysis, samples (20 μl) were mixed with 7.34 μl of 4× SDS gel loading buffer (200 mM Tris-HCl, pH 6.8, 8% SDS, 20% Glycerol, 0.4% bromophenol blue and 400 mM β-mercaptoethanol) and 2 μl of cell lysing buffer, boiled for 10 min, cooled on ice, spun on a microcentrifuge and transferred to wells of an electrophoresis apparatus containing a 4% acrylamide stacking gel and a 8% acrylamide resolving gel. Electrophoresis protein separation was performed overnight using a constant current (40 V). Proteins were transferred to nitrocellulose by electro-blotting in a 20% methanol-transfer buffer with a constant 900 mA current for 90 min. Ponceau Red staining was performed to evaluate the quality of loading and transfer. Then, the membrane was blocked with TBS-[5% BSA-0.1% Tween™] for 1 h at room temperature, probed with a mouse IgG1 Anti-Tau 46 (Santa Cruz, Cat. No. SC-32274, 1/150 in TBS-0.1%Tween™) at 4° C. overnight, washed 4 times for 5 min each time with TBS-0.1% Tween™, and probed with an anti-Mouse IgG-peroxidase antibody (Sigma Cat. No. A2554, 1/3000 in TBS-[5% skimmed milk-0.1% Tween™] for 1 h at room temperature). The membrane was then washed 4 times for 5 min each time with TBS-0.1% Tween™, and soaked with the chemiluminescence substrate (Enhanced Luminol™ from PerkinElmer). X-Ray films were exposed to the membranes long enough to optimize each band detection. Band Intensity was quantified using a Fluor-S Multimager™ (Bio-Rad scanner) and the Quantity One™ software (Bio-Rad) and data adjusted with local background.

In parallel, on day 9, plates were used for the MTT cell viability assay. A volume of 62.5 μl of a 3-(4,5-Dimethylthiazol-2-yl)-2,5-diphenyltetrazolium bromide (MTT) solution (5 mg/ml in a 1:1 PBS/culture medium) was added, then plates were incubated for 4 h. A volume of 312.5 μl of isopropanol—0.1N HCl was added, then for each well the content was mixed using a pipette (wide bore tip) to solubilize the product. The optical density at 560 nm, which is proportional to the number of living cells, was measured (reference at 690 nm).

### EXAMPLE 2

### Effect of Curcumin and Everolimus on Tau Protein Levels in Differentiated SH-SY5Y Cells

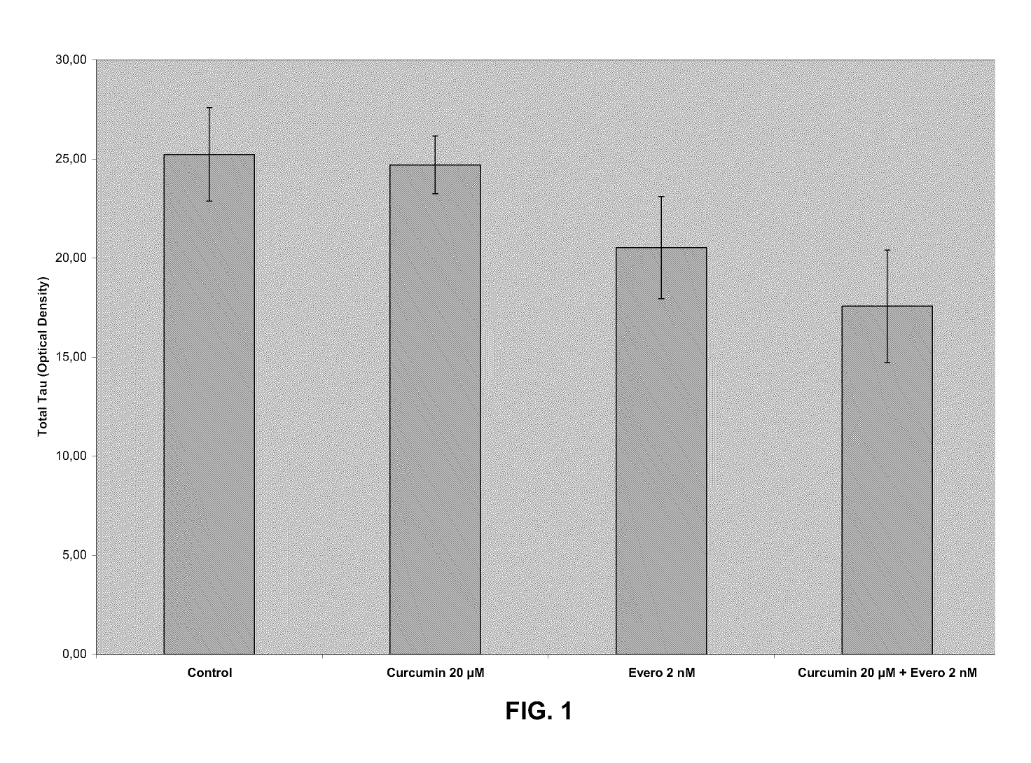

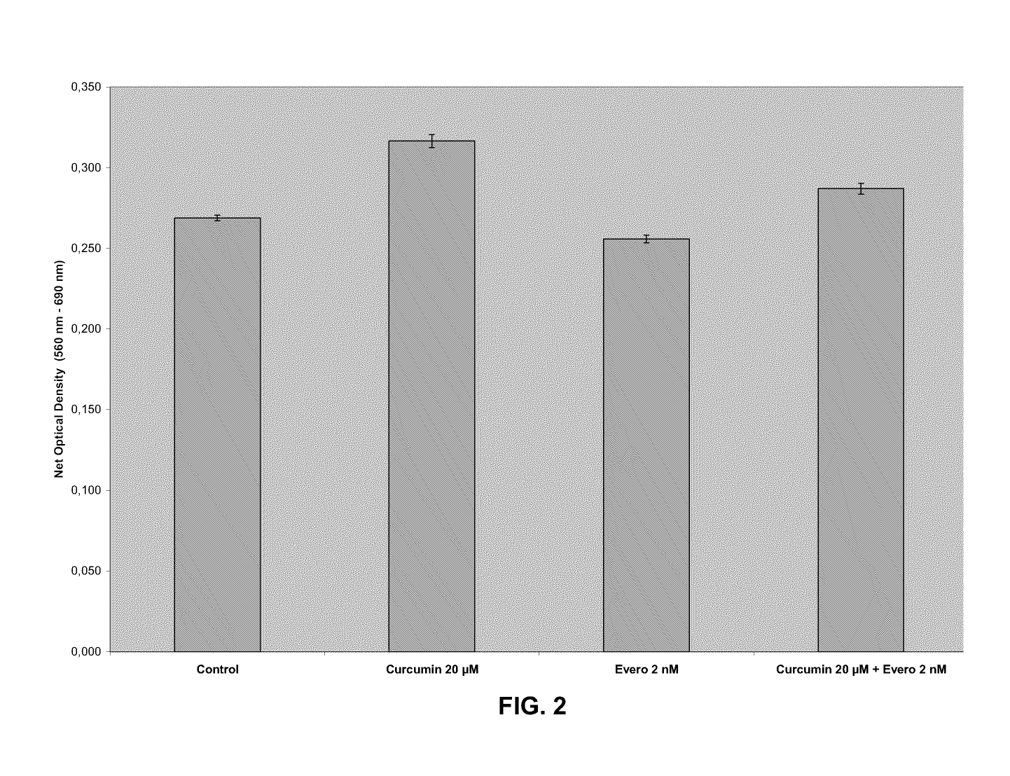

The effect of curcumin and/or Everolimus on the levels of Tau protein in differentiated SH-SY5Y human neuron-like cells was determined. A 48 h treatment with 20 μM curcumin in the presence of 5 nM human β-amyloid₍₁₋₄₂₎, induced a 2.1% reduction in the total amount of Tau protein as compared to controls. In comparison, cells treated with 2 nM Everolimus showed a 18.7% reduction in the total amount of Tau protein, whereas cells treated with a combination of 20 μM curcumin and 2 nM Everolimus showed a synergistic reduction of 30.3%, i.e., 9.5% greater than the sum of the two treatments alone, which represents a 45.7% increase compared to the sum of the individual effects (FIG. 1). As shown in FIG. 2, none of these three treatments were cytotoxic.

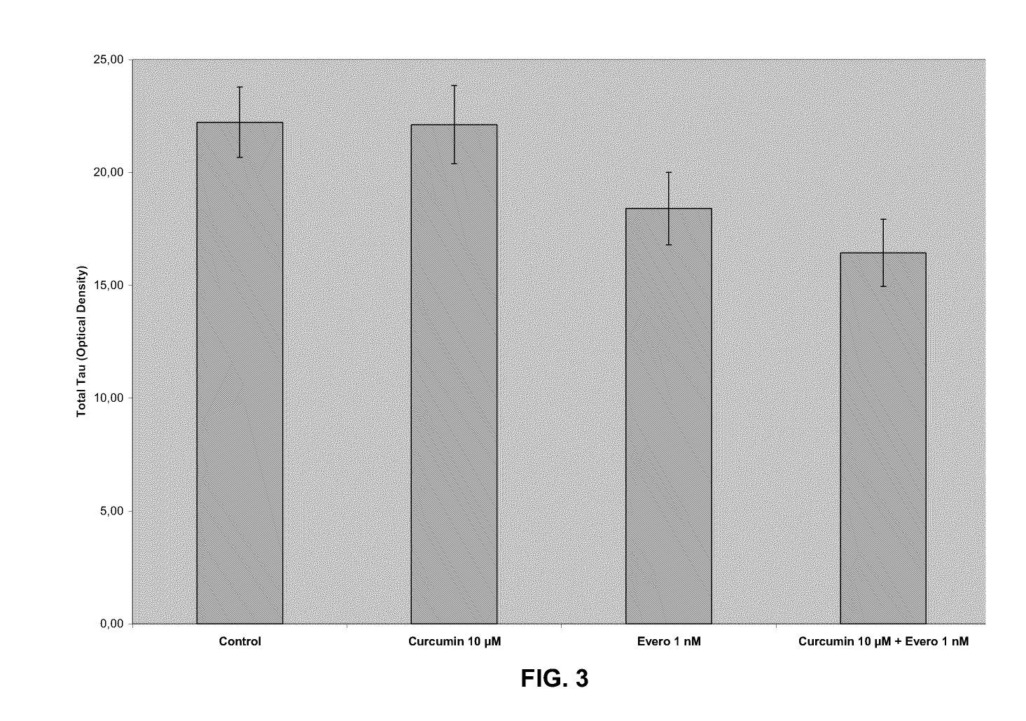

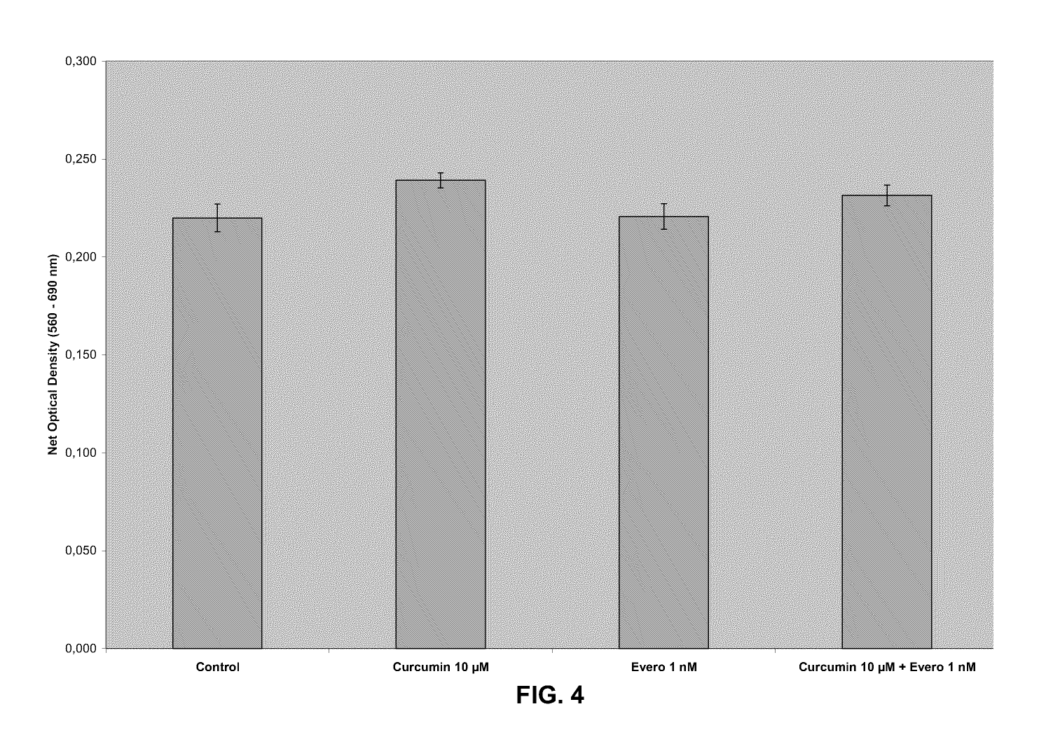

Similarly, cells treated with 10 μM curcumin for 48 h in the presence of 5 μM human β-amyloid₍₁₋₄₂₎ showed a 0.5% reduction in the total amount of Tau protein as compared to controls. Cells treated with 1 nM Everolimus showed a 17.2% reduction in the total amount of Tau protein, whereas cells treated with the combination of 10 μM curcumin and 1 nM Everolimus showed a synergistic reduction of 26.0%, i.e. 8.3% greater than the sum of the two treatments alone, which represents a 46.9% increase compared to the sum of the individual effects (FIG. 3). Again, none of these three treatments were cytotoxic (FIG. 4).

These data show that curcumin and Everolimus act synergistically to reduce the levels of Tau proteins in neuron-like cells treated with a human β-amyloid peptide, a model which mimics the features of Alzheimer's disease (Lambert et al., J Neurosci Res. 1994 39(4): 377-85; Jämsä et al. 2004, supra), and do not show significant cell toxicity.

### EXAMPLE 3

### Effect of Curcumin and Temsirolimus on Tau Protein Levels in Differentiated SH-SY5Y Cells

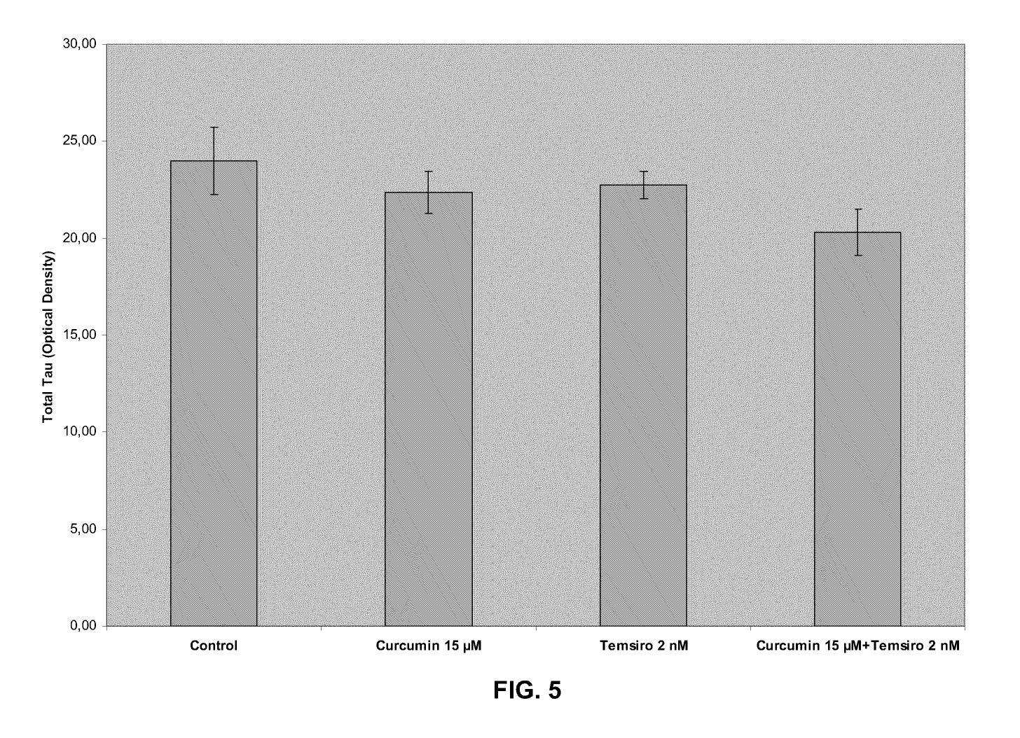

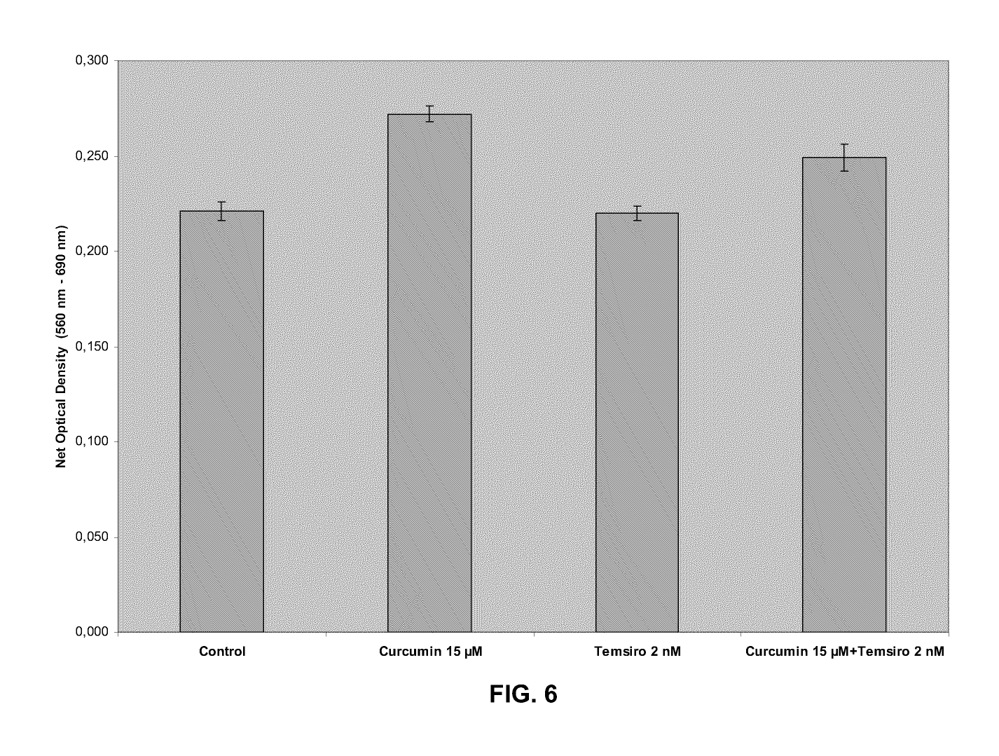

The effect of curcumin and/or Temsirolimus on the levels of Tau protein in differentiated SH-SY5Y human neuron-like cells was determined. A 48 h treatment with 15 μM curcumin in the presence of 5 nM human β-amyloid₍₁₋₄₂₎, induced a 6.7% reduction in the total amount of Tau protein as compared to controls. In comparison, cells treated with 2 nM Temsirolimus showed a 5.3% reduction in the total amount of Tau protein, whereas cells treated with a combination of 15 μM curcumin and 2 nM Temsirolimus showed a synergistic reduction of 15.4%, i.e., 3.4% greater than the sum of the two treatments alone, which represents a 28.3% increase compared to the sum of the individual effects (FIG. 5). As shown in FIG. 6, none of these three treatments were cytotoxic.

These data show that curcumin and Temsirolimus act synergistically to reduce the levels of Tau proteins in neuron-like cells treated with a human β-amyloid peptide, a model which mimics the features of Alzheimer's disease (Lambert et al., J Neurosci Res. 1994 39(4): 377-85; Jämsä et al. 2004, supra), and do not show significant cell toxicity.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method of treating a tauopathy in a subject, said method comprising administering a curcuminoid and a mammalian target of rapamycin (mTOR) inhibitor to said subject.

2. The method of claim 1, wherein said curcuminoid is curcumin or a derivative thereof.

3. The method of claim 1, wherein said curcuminoid is curcumin.

4. The method of claim 1, wherein said mTOR inhibitor is a rapamycin derivative.

5. The method of claim 4, wherein said rapamycin derivative is Everolimus or Temsirolimus.

6. The method of claim 1, wherein said tauopathy is Alzheimer's disease.

7-8. (canceled)

9. The method of claim 1, wherein said curcuminoid and said mTOR inhibitor are administered simultaneously.

10. The method of claim 1, wherein said curcuminoid and said mTOR inhibitor are administered sequentially.

11. The method of claim 1, wherein said method comprises administering a composition comprising said curcuminoid and said mTOR inhibitor.

12. The method of claim 1, wherein said method comprises administering a first composition comprising said curcuminoid and a pharmaceutically acceptable carrier, and a second composition comprising said mTOR inhibitor and a pharmaceutically acceptable carrier.

13. A method of decreasing the level of Tau protein in a cell, tissue or organ, said method comprising contacting said cell, tissue or organ with a curcuminoid and an mTOR inhibitor.

14. The method of claim 13, wherein said curcuminoid is curcumin or a derivative thereof.

15. The method of claim 14, wherein said curcuminoid is curcumin.

16. The method of claim 13, wherein said mTOR inhibitor is a rapamycin derivative.

17. The method of claim 16, wherein said rapamycin derivative is Everolimus or Temsirolimus.

18. The method of claim 13, wherein said cell, tissue or organ is a neuronal cell, tissue or organ.

19. A kit comprising a curcuminoid, an mTOR inhibitor, and instructions for treating a tauopathy in a subject, or for decreasing the level of Tau protein in a cell, tissue or organ.

20. The kit of claim 19, wherein said curcuminoid is curcumin or a derivative thereof.

21. (canceled)

22. The kit of claim 19, wherein said mTOR inhibitor is a rapamycin derivative.

23. The kit of claim 22, wherein said rapamycin derivative is Everolimus or Temsirolimus.

24-52. (canceled)
